(19) United States
(12) Patent Application Publication
(10) Pub. No.: US 2005/0177231 A1
(43) Pub. Date: Aug. 11, 2005

(54) Intra-ocular implant promoting direction guided cell growth

(76) Inventors: John L. Ricci, Middletown, NJ (US); Harold Alexander, Short Hills, NJ (US)

Correspondence Address: MELVIN K. SILVERMAN, 500 WEST CYPRESS CREEK ROAD, SUITE 500, FT. LAUDERDALE, FL, 33309, US

(21) Appl. No.: 11/042,249

(22) Filed: Jan. 24, 2005

Related U.S. Application Data
(63) Continuation-in-part of application No. 10/241,256, filed on Sep. 10, 2002, now abandoned.
(60) Provisional application No. 60/317,830, filed on Sep. 10, 2001.

(51) Int. Cl. A61F 2/16; A61F 2/14

(52) U.S. Cl. 623/6.16; 623/5.15

(57) ABSTRACT

An intra-ocular implant has an optical portion capable of optically transmitting light from an exterior of an eye to a retina thereof; and an outer skirt portion extending annularly about the optical portion. The outer skirt portion has anterior and posterior surfaces, one or both of the surfaces having a multiplicity of alternating regular microgrooves and ridges extending annularly about the optical portion. The multiplicity of alternating regular microgrooves and ridges have a substantially same width in a range of between about 4 to about 40 micrometers and a substantially same depth in a range of between about 4 to about 40 micrometers. The multiplicity of alternating regular microgrooves and ridges promote cell growth in a direction along longitudinal axes of the annular microgrooves and inhibition of cell growth in a direction perpendicular to the longitudinal axes, thereby inhibiting cell growth into the optical portion.

### CROSS REFERENCE TO RELATED APPLICATION

This application is continuation-in-part of patent application Ser. No. 10/241,256, filed on Sep. 10, 2002, which claims the benefit under 35 USC 119 (e) of the provisional patent application Ser. No. 60/317,830, filed Sep. 10, 2001. All prior application is hereby incorporated by reference in their entireties.

### BACKGROUND OF THE INVENTION

Intra-ocular implants, typically in the form of a corneal prosthesis, are known in the art, as is reflected in such references as U.S. Pat. No. 5,489,301 (to Barber), entitled Corneal Prosthesis; and U.S. Pat. No. 6,106,552 (to Lacombe, et al), entitled Corneal Prosthesis Device Having Anterior and Posterior Annular Skirts. Such intra-ocular implants address a range of opthalmatic issues, including aphakia (the absence of a lens).

Historic problems in the use of intra-ocular implants in apikaratoplasty or epikeiratophakia have been that of assuring the stability of the implant relative to the region of the eye upon which it is to be secured, and the prevention of epithelial overgrowth thereby causing opacity. As is apparent, any movement or dislocation of an intra-ocular implant from its desired placement can have consequences which are at least adverse and often disastrous in terms of the success of a given procedure. Likewise, opacity due to overgrowth can severely compromise the success of the implant procedure.

U.S. Pat. No. 4,808,181 (to Kelman) teaches an intraocular lens having roughened surface area. The intraocular lens has a portion of the posterior surface formation constituting a planar contact region to be seated against the tissue. This planar contact region is provided with a roughened surface area for accelerated adhesion of the tissue of the adjacent posterior capsule part to the depressions and enhanced anchoring of the lens to the posterior capsule part. Kelman specifically teaches that the roughened surface area can be either defined by a series of ordered narrow linear depressions extending transversely of the plane of contact region, or in the shape of individual spaced apart segment of an interrupted ring or annulus extending around the optic. The roughened surface area is also disposed on the pair of haptic. Kelman teaches that the roughened surface area depressions are of a depth of at least about 0.01 mm to about 0.12 mm, with no requirement on the width of the depressions specified.

U.S. Pat. No. 5,549,670 (to Young et al) teaches an intraocular lens for reducing secondary opacification. The intraocular lens has an optical portion, a cell barrier portion circumscribes the optical portion and an elongated fixation member. The cell barrier portion includes irregularly configured structure, for example, irregular grooves and is incapable of focusing light on the retina. Young et al specifically teach that the grooves are substantially completely defined by irregular surfaces. Furthermore, Young et al teach that the irregularly configured structure of cell barrier portion, such as the irregular grooves, acts to disrupt or otherwise interfere with the process of eye cell, for example, lens epithelial cell, migration or growth, so that the cumulative effect of this irregular structure is to significantly reduce, or even eliminate, the migration or growth of cells in front of or in back of the optical portion after the intraocular lens is implanted in the eye.

Even with these known efforts, however, currently epithelial overgrowth into the visual areas is still a major clinical problem. Therefore, it is desirable to have improved intraocular lens which provides effective inhibition of cell growth into the visual area of the optic.

### SUMMARY OF THE INVENTION

In one embodiment, the present invention is directed to an intra-ocular implant which comprises an optical portion capable of optically transmitting light from an exterior of an eye to a retina thereof; and an outer skirt portion extending annularly about the optical portion, the outer skirt portion having anterior and posterior surfaces, at least one of the surfaces having a multiplicity of alternating regular microgrooves and ridges extending annularly about the optical portion. The multiplicity of alternating regular microgrooves and ridges have a substantially same width in a range of between about 4 to about 40 micrometers and a substantially same depth in a range of between about 4 to about 40 micrometers. The multiplicity of alternating regular microgrooves and ridges promote cell growth in a direction along longitudinal axes of the microgrooves and inhibition of cell growth in a direction perpendicular to the longitudinal axes, thereby inhibiting cell growth into the optical portion.

In a further embodiment, both anterior and posterior surfaces of the outer skirt portion have a multiplicity of alternating regular microgrooves and ridges extending annularly about the optical portion.

It is an object of the present invention to provide an intra-ocular implant having means of preventing epithelial overgrowth into the optical portion of the implant.

It is a further object of the invention to provide an intro-ocular implant of the above type having application in various post operative and traumatic conditions of the eye.

The above and yet other objects and advantages of the invention will become apparent from the hereinafter-set forth Brief Description of the Drawings and Detailed Description of the Invention and claims appended herewith.

### BRIEF DESCRIPTION OF THE DRAWINGS

FIG. 1 is a front elevational view of an intra-ocular implant in accordance with the present invention.

FIG. 2 is a cross-sectional view taken along Line 2-2 of FIG. 1.

FIG. 3 is an enlarged sectional view showing a part of the anterior surface of the outer skirt portion at the interface between the optical portion and the outer skirt portion shown in FIG. 1.

FIG. 4 is an enlarged cross-sectional view along Line 3-3 of FIG. 3 showing the anterior and the posterior surfaces of the outer skirt portion.

FIGS. 5A to 5H are cross-sectional views of various configurations of the microgrooves and ridges, which can be used on the anterior and posterior surfaces of the outer skirt portion of the intra-ocular implant of the present invention.

### DETAILED DESCRIPTION OF THE INVENTION

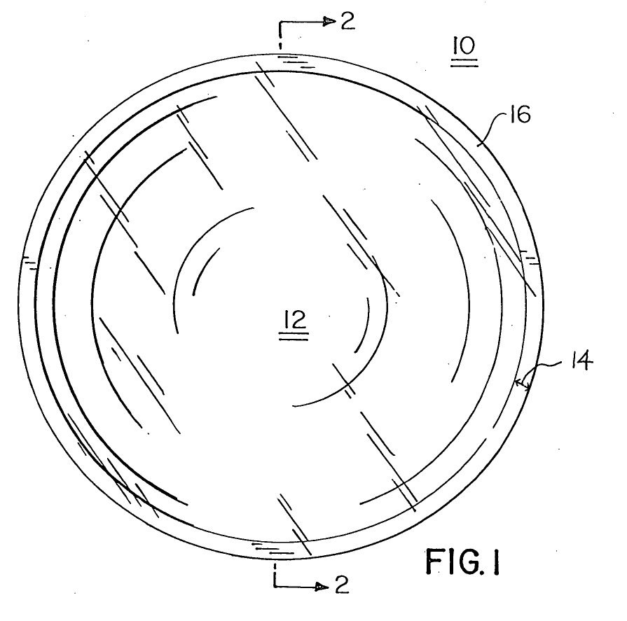

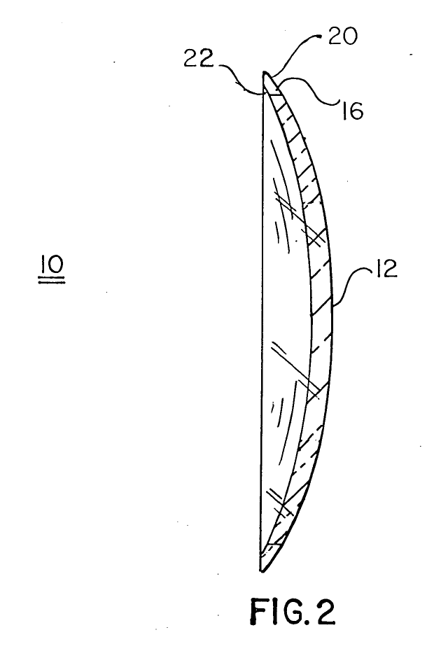

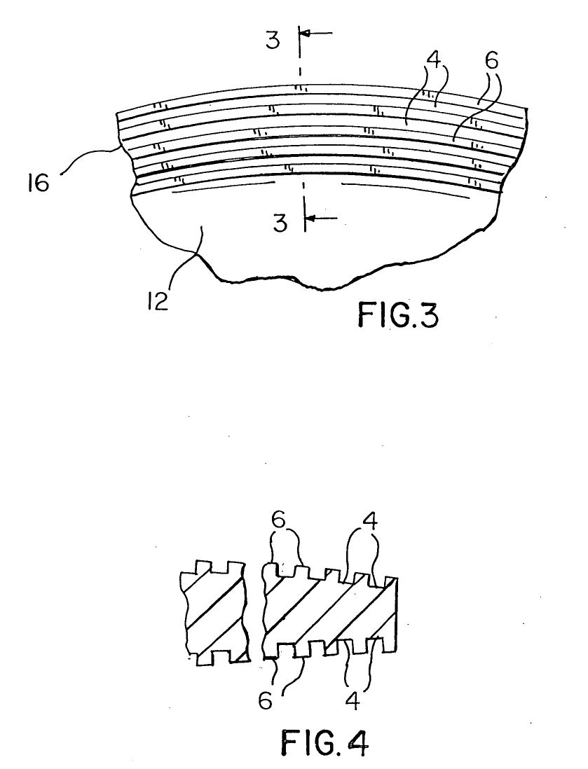

The present invention provides an intra-ocular implant which induces direction guided cell growth of the surrounding tissue. As shown in FIGS. 1-2, the intra-ocular implant 10, as a corneal implant, has an arcuate optical portion 12 capable of optically transmitting light from an exterior of an eye to a retina of the eye; and an outer skirt portion 16 extending annularly about the optical portion 12. The outer skirt portion 16 has an anterior surface 20 and a posterior surface 22. One or both of the anterior surface 20 and posterior surface 22 have a multiplicity of alternating regular microgrooves 4 and ridges 6 extending annularly about the optical portion 12, as shown in FIGS. 3 and 4. It is noted that FIG. 3 shows an enlarged view of only a part of the anterior surface of the outer skirt portion at the interface between the optical portion 12 and the outer skirt portion 16 for illustration of the regular microgrooves 4 and ridges 6. More alternating regular microgrooves 4 and ridges 6 extending outwardly from the optical portion are not shown.

Herein, the term “microgroove” refers to a groove having a width and a depth in the order of micrometers, more particularly having a width and a depth less than 100 micrometers. The term “regular” herein denotes controlled structural features including shape, dimension and surface condition, which are substantially free irregular structural patterns.

Preferably, the multiplicity of microgrooves 4 have a substantially same width in a range of between about 4 to about 40 micrometers, and a substantially same depth in a range of between about 4 to about 40 micrometers. More preferably, the width and the depth of the microgrooves 4 are in a range of between about 6 to about 28 micrometers. As described hereinafter in detail, these regular and repetitive microgrooves promote cell growth in a direction along longitudinal axes of the microgrooves and inhibition of cell growth in a direction perpendicular to the longitudinal axes. Such guided cell growth assists integration and stabilization of the implant into the surrounding tissue, and at the same time inhibits cell growth into the optical portion 12.

The intra-ocular implant can be made of an optical polymeric material, which is biocompatible with the tissue of the human eye and capable of receiving vascular invasion and formation of fibrous tissue therein during the healing process. Suitable materials for producing the intra-ocular implant of the present invention include, but are not limited to, the commonly used materials for rigid optical lens, such as polymethylmethacrylate, or commonly used for deformable lens, such as silicone polymeric materials, acrylic polymeric materials, porous polytetraflouroethylene, polyhydroxyethylmethacrylate and mixtures thereof.

The outer skirt portion 16 of the intra-ocular implant 10 corresponds to the peripheral portion of a conventional implant. The radial width 14 of the outer skirt portion 16 can be about 5% to about 25% of the radius of the implant. Preferably, there are at least 20 microgrooves 4 in the outer skirt portion 16.

In FIGS. 1 and 2, the optical portion and the intra-ocular implant are circular. However, it should be understood that the optical portion of the implant can also have other suitable shapes, and the outer skirt portion can surround and extend annularly about the optical portion in a corresponding shape.

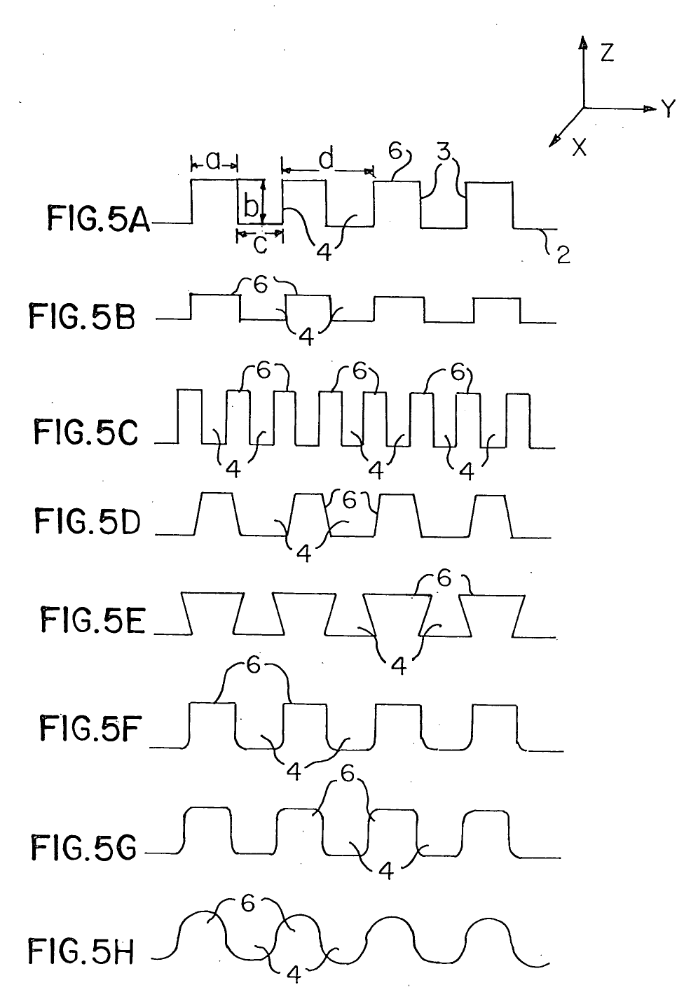

FIGS. 5A to 5H illustrate cross-sectional views of various configurations of alternating regular microgrooves and ridges that can be used for the intra-ocular implant of the present invention. As shown in FIGS. 5A-5H, on a substantially flat surface there are a multiplicity of alternating microgrooves 4 and ridges 6. The alternating microgrooves 4 and ridges 6 extend along their longitudinal axes (in the direction of X-axis, as shown in FIG. 5A). Each microgroove has a groove base 2 and two opposing groove walls 3. The dimensions of the microgrooves 4 and ridges 6 are indicated by the letters “a”, “b”, “c” and “d”. More specifically, “a” is the width of ridges 6, “c” is the width of the microgrooves 4, “b” is the depth of the microgrooves 4, and d is the spacing (or pitch) between adjacent ridges 6. The configuration shown in FIG. 5A has square ridges 6 and square microgrooves 4, where “a”, “b” and “c” are equal and the spacing d between adjacent ridges 6 is twice that of “a”, “b” and “c”. FIGS. 5B and 5C illustrate two rectangular configurations where the depth b is not equal to that of “a” and/or “c”.

FIGS. 5D and 5E illustrate trapezoidal configurations formed by microgrooves 4 and ridges 6 where the angles formed by one groove wall 3 and groove base 2 can be either greater than 90° as shown in FIG. 5D or less than 90° as shown in FIG. 5E. As shown in the above-configurations, the angle formed by the groove wall 3 and groove base 2 is in a range from about 60 degree to about 120 degree.

In FIG. 5F, the corners formed by the intersection of the groove wall 3 and groove base 2 have been rounded, and in FIG. 5G, these corners as well as the corners formed by the intersection of the surface of the ridge and the groove wall 3 have been rounded. These rounded corners can range from arcs of only a few degrees to arcs where consecutive microgrooves 4 and ridges 6 approach the configuration of a sine curve as shown in FIG. 5H.

In the microgrooves illustrated in FIGS. 5A to 5H, the width of the microgrooves 4, can be from about 6 to about 40 μm (micrometers), and preferably from about 10 μm to about 28 μm. In the trapezoidal configurations as shown in FIGS. 5D and 5E, the width of the microgrooves can be defined as the width at the half height of the microgroove. The width of the ridge, can be equal or different from the width of the microgroove depending on the design needs. The depth of the microgroove is preferably similar to the width of the microgroove for the purpose of inhibiting epithelial overgrowth. As shown, the surfaces of the groove walls and groove base are substantially flat.

The above-described microgrooves can be produced on the anterior and posterior surfaces of the outer skirt by laser ablation techniques known in the art, for example, the instrument and methodology illustrated in details in U.S. Pat. Nos. 5,645,740 and 5,607,607, which are herein incorporated by reference in their entireties. Using laser ablation, the width and depth of the microgrooves can be produced with an error range of less than 0.01 μm. Therefore, a specific configuration of alternating microgrooves and ridges can be selected based on the need, and controllably produced to provide a multiplicity of microgrooves have a substantially same width and a substantially same depth. Furthermore, with the precision of the laser ablation technology, the surfaces of the groove walls 3 and groove base 2 are substantially planer, and free of random surface structures. Alternatively, the multiplicity of alternating regular microgrooves 4 and ridges 6 can be produced by molding, using the methods known in the art. With molding, the surfaces of the groove walls 3 and groove base 2 are smoother, and the surface variations in the Y-axis for the groove walls 3, or in the Z-axis for the groove base 2 are in the magnitude of Angstroms.

The above-described alternating microgrooves and ridges produced on the anterior and posterior surfaces of the outer skirt portion of the intra-ocular implant can be utilized to promote guided tissue attachment, and at the same time to inhibit epithelial overgrowth into the optical portion. The effectiveness in suppression of overall cell growth on a cell culture surface having the above-described alternating microgrooves and ridges have been described in U.S. Pat. Nos. 5,645,740, 5,607,607 and 6,419,491, which are herein incorporated by reference in their entireties.

More specifically, as described in U.S. Pat. No. 5,645,740 using a titanium oxide surface with the microgrooves and ridges shown in Table 1, a substantial suppression of rat tendon fibroblast (RTF) cell growth was observed in comparison with the control which grew the same type of cells on a flat smooth surface.

TABLE 1

| | Actual Dimension (μm) |
|---|---|
| Configuration | (a × c × b) |
| 2 μm | 1.80 × 1.75 × 1.75 |
| 4 μm | 3.50 × 3.50 × 3.50 |
| 6 μm | 3.50 × 3.50 × 3.50 |
| 8 μm | 8.00 × 7.75 × 7.50 |
| 12 μm | 12.00 × 11.50 × 7.5 |

Note:
To simplify nomenclature, the configuration used in these studies are referred to as 2 μm (a = 1.80 μm), 4 μm (a = 3.50 μm), 6 μm (a = 6.50 μm), 8 μm (a = 8.00 μm), and 12 μm (a = 12.00 μm).

The surface having the alternating microgrooves and ridges were observed to result in elongated colony growth in the direction along the longitudinal axes (also referred to as X-axis) of the microgrooves 4 and inhibition of cell growth in the direction perpendicular to the longitudinal axes (also referred to as Y-axis) of the microgrooves 4. On an individual cell level, the cells had elongated morphology and appeared to be “channeled” along the microgrooves, as compared with control culture where outgrowing cells move randomly on flat surfaces. The most efficient “channeling” was observed with the 6 μm and 8 μm microgrooves. With 6 μm and 8 μm microgrooves, the rat tendon fibroblast cells were observed to attach and orient within the microgrooves. This rendered almost no growth in the Y-axis on the planar surface having these microgrooves.

On the surface having smaller microgrooves and ridges, a different effect was observed. The RTF cells bridged the surfaces having the 2 μm microgrooves resulting in cells with different morphologies than those surfaces having the 6, 8, and 12 μm microgrooves. These cells were wide and flattened and were not well oriented. On the surface having 4 μm microgrooves, the RTF cells showed mixed morphologies, with most cells aligned and elongated but not fully attached within the microgrooves. This resulted in appreciable growth of the RTF cells in the Y-axis on the surfaces having 2 and 4 μm microgrooves. On the other hand, limited Y-axis growth was observed when the RTF cells were grown on the surface having 12 μm microgrooves.

The results of the observed effects of these surfaces having alternating microgrooves and ridges on overall RTF cell colony growth were pronounced. All above-described tested surfaces caused varying but significant increases in X-axis growth compared to the controls, and varying but pronounced inhibition of Y-axis growth. More importantly, this resulted in suppression of overall growth of the RTF cell colony compared with the control. It was also shown that the suppression of cell growth differed between different types of cells.

As shown in FIG. 3 and described above, the alternating microgrooves 4 and ridges 6 on the anterior and posterior surfaces of the outer skirt portion 16 of the intra-ocular implant extend annularly about the optical portion 12. It should be understood, therefore, that the longitudinal axis of each microgroove also extends annularly about the optical portion 12, and the direction perpendicular to the longitudinal axis is in the radial direction from the center of the optical portion 12. With this structural arrangement, the alternating microgrooves 4 and ridges 6 on the anterior and posterior surfaces of the outer skirt portion guide the cell growth about the optical portion, and inhibit the cell growth into the optical portion of the implant.

As can be appreciated, the intra-ocular implant 10 has medical application not only as a corneal prosthesis but, as well, in a variety of post-operative and post-traumatic situations wherein less than permanent coverage of a portion of the eye is desirable. In such application, eventual removal of the implant may be readily facilitated through the use of laser means of a type now commonly used in ocular surgery.

While there has been shown and described the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention as set forth herewith.

1. An intra-ocular implant comprising:
an optical portion capable of optically transmitting light from an exterior of an eye to a retina thereof; and
an outer skirt portion extending annularly about said optical portion, said outer skirt portion having anterior and posterior surfaces, at least one of said surfaces having a multiplicity of alternating regular microgrooves and ridges extending annularly about said optical portion; said multiplicity of alternating regular microgrooves and ridges having a substantially same width in a range of between about 4 to about 40 micrometers and a substantially same depth in a range of between about 4 to about 40 micrometers;
wherein said multiplicity of alternating regular microgrooves and ridges promote cell growth in a direction along longitudinal axes of said microgrooves and inhibit cell growth in a direction perpendicular to said longitudinal axes, thereby inhibiting cell growth into said optical portion.

2. The intra-ocular implant of claim 1, wherein said width and depth of said microgrooves are in a range of between about 6 to about 28 micrometers.

3. The intra-ocular implant of claim 1, wherein said microgrooves are produced by laser ablation, or molding.

4. The intra-ocular implant of claim 1, wherein each of said microgrooves has a groove base and two opposing groove walls, and said groove base and walls have substantially flat surfaces.

5. The intra-ocular implant of claim 1, wherein said implant is a corneal prosthesis.

6. The intra-ocular implant of claim 1, wherein said implant is for epikeratophakia.

7. An intra-ocular implant comprising:
an optical portion capable of optically transmitting light from an exterior of an eye to a retina thereof; and
an outer skirt portion extending annularly about said optical portion, said outer skirt portion having anterior and posterior surfaces, each of said surfaces having a multiplicity of alternating regular microgrooves and ridges extending annularly about said optical portion; said multiplicity of alternating regular microgrooves and ridges having a substantially same width in a range of between about 4 to about 40 micrometers and a substantially same depth in a range of between about 4 to about 40 micrometers;
wherein said multiplicity of alternating regular microgrooves and ridges promote cell growth in a direction along longitudinal axes of said microgrooves and inhibit cell growth in a direction perpendicular to said longitudinal axes, thereby inhibiting cell growth into said optical portion.

8. The intra-ocular implant of claim 7, wherein said width and depth of said microgrooves are in a range of between about 6 to about 28 micrometers.

9. The intra-ocular implant of claim 7, wherein said microgrooves are produced by laser ablation, or molding.

10. The intra-ocular implant of claim 7, wherein each of said microgrooves has a groove base and two opposing groove walls, and said groove base and walls have substantially flat surfaces.

11. The intra-ocular implant of claim 7, wherein said implant is a corneal prosthesis.

12. The intra-ocular implant of claim 7, wherein said implant is for epikeratophakia.
